(19) United States
(12) Patent Application Publication
(10) Pub. No.: US 2026/0272335 A1
(43) Pub. Date: Sep. 17, 2026

(54) CUSTOMIZED WEARABLE SALIVA BIOSENSOR FOR REAL-TIME CONTINUOUS HEALTH MONITORING

(71) Applicant: The Hong Kong Polytechnic University, Hung Hom (HK)

(72) Inventors: Feng YAN, Hung Hom (HK); Zeyu ZHAO, Hung Hom (HK)

(73) Assignee: The Hong Kong Polytechnic University, Hung Hom (HK)

(21) Appl. No.: 19/658,214

(22) Filed: Apr. 24, 2026

Related U.S. Application Data
(60) Provisional application No. 63/764,282, filed on Feb. 27, 2025.

(51) Int. Cl. A61B 5/145; A61B 5/00; A61B 5/1477; A61B 5/1486

(52) U.S. Cl. CPC A61B 5/14507; A61B 5/14532; A61B 5/14546; A61B 5/1477; A61B 5/1486; A61B 5/682; A61B 5/742; A61B 2560/0214; A61B 2562/125; A61B 2562/166

(57) ABSTRACT

A customized wearable saliva biosensor system for continuous, real-time, in-situ monitoring of biochemical markers in human saliva. The system integrates a multi-gate organic electrochemical transistor biosensor employing an organic semiconductor channel with enzyme-functionalized gate electrodes, a compact wireless integrated circuit with Bluetooth data transmission, a power subsystem optionally incorporating piezoelectric and/or triboelectric nanogenerators, and a custom-fitted wearable substrate fabricated from a three-dimensional scan of the user's dental anatomy. The system is positioned within the oral cavity in proximity to the sublingual glands for direct detection of fresh saliva at the point of secretion. A differential measurement architecture comparing an enzyme gate to a control gate provides selective, real-time detection of target biomarkers including glucose, lactate, and uric acid. Measured data is wirelessly transmitted to a companion mobile application for display to the user.

### CROSS-REFERENCE TO RELATED APPLICATIONS

This application claims priority from and the benefit of U.S. Provisional Patent Application 63/764,282, titled “Customized and Comfortable Real-Time Saliva Biosensors for Continuous Health Monitoring” filed Feb. 27, 2025 in the United States Patent & Trademark Office. All disclosures of the document(s) named above are incorporated herein by reference.

### BACKGROUND OF THE INVENTION

### 1. Field of the Invention

Aspects of the invention relate to a wearable biosensor system for continuous, real-time, in-situ monitoring of biochemical markers in human saliva.

### 2. Description of the Related Art

Continuous monitoring of biochemical markers in biological fluids is an important tool for health management, disease detection, and human performance monitoring. Blood-based monitoring, while well-established, is invasive and impractical for continuous real-time use outside clinical settings.

Saliva is an accessible biological fluid that contains meaningful concentrations of metabolites including glucose, lactate, and uric acid. These metabolites reflect physiological states including metabolic activity, nutritional status, and exertion level. Salivary biomarker concentrations correlate with blood concentrations of the same markers, though with characteristic time delays that vary by individual and by activity.

Existing saliva-based biosensor devices generally require the collection of a saliva sample outside the oral cavity for analysis. This sample-collection approach introduces delays, risks contamination, and does not permit continuous real-time monitoring.

Wearable biosensors placed within the oral cavity offer the potential for continuous, in-situ monitoring of salivary biomarkers without the need for external sample collection. However, existing intraoral devices suffer from significant drawbacks including poor fit and comfort, limited sensitivity, lack of wireless data transmission, and inability to monitor multiple biomarkers simultaneously.

A non-invasive wearable biosensor system capable of continuous, real-time, simultaneous monitoring of multiple salivary biomarkers directly within the oral cavity, with wireless data transmission to an external device, would be a useful invention.

A wearable biosensor system of this type that is customized to the individual user's oral anatomy for comfort and secure fit would be a useful invention.

Aspects of the present invention address these and other concerns.

### SUMMARY OF THE INVENTION

Aspects of the invention relate to a customized wearable saliva biosensor system for continuous, real-time, in-situ monitoring of biochemical markers in human saliva. The system integrates a multi-gate organic electrochemical transistor (“OECT”) biosensor, a wireless integrated circuit, an ultrathin power subsystem, and a customized wearable substrate, forming a compact functional sensing platform suitable for intraoral use.

The OECT biosensor employs a patterned organic semiconductor channel with gate electrodes functionalized with enzyme-specific coatings, enabling selective detection of target biomarkers at low concentrations. A differential measurement architecture using an enzyme-functionalized gate and a control gate improves selectivity and reduces interference from non-target analytes.

The wireless integrated circuit enables real-time data transmission to a paired smartphone or other external device. A companion mobile application converts the measured channel current response into corresponding biomarker concentration values.

The power subsystem incorporates a suitable power source and may additionally incorporate piezoelectric and/or triboelectric nanogenerators to extend operational life and reduce maintenance requirements.

The wearable substrate is custom-fabricated based on a three-dimensional scan of the individual user's dental anatomy, providing a secure and comfortable fit. The substrate incorporates a self-adaptive fitting mechanism and may employ machine learning algorithms to continuously refine the fit based on user feedback and wear data.

The biosensor is positioned within the wearable substrate in proximity to the sublingual glands, enabling direct detection of fresh saliva at the point of secretion.

Additional aspects and/or advantages of the invention will be set forth in part in the description which follows and, in part, will be obvious from the description, or may be learned by practice of the invention.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 is a schematic diagram of the layered structure of the coin-sized wireless integrated circuit of one embodiment of the invention, showing the substrate, battery, circuit, socket, OECT, and PDMS layers.

FIG. 2 is an illustration and photograph of the wearable brace integrating the biosensor system for continuous intraoral salivary monitoring.

FIG. 3 is a schematic illustration of the multi-gate OECT design of one embodiment of the invention.

FIG. 4 is a schematic diagram and photograph showing placement of the biosensor system within the human oral cavity.

FIG. 5 is a diagram illustrating the potential drop between the gate electrode and the OECT channel in a metabolite solution, comparing the enzyme gate response to the control gate response.

FIG. 6 is a series of graphs providing continuous monitoring data for glucose, uric acid, and lactate in saliva and blood across four daily activities: eating, drinking, running, and office work.

FIG. 7 is a series of graphs providing clinical trial data for fasting and continuous salivary glucose monitoring across multiple sessions and volunteers.

FIG. 8 is an abstract schematic of one complete embodiment of the wireless data processing and output components of the invention.

### DETAILED DESCRIPTION OF THE EMBODIMENTS

Reference will now be made in detail to several embodiments of the invention that are illustrated in the accompanying drawings. Whenever possible, the same or similar reference numerals are used in the drawings and the description to refer to the same or like parts or steps. The drawings are in simplified form and are not to precise scale. For purposes of convenience and clarity only, directional terms such as top, bottom, left, right, up, down, over, above, below, beneath, rear, and front, can be used with respect to the drawings. These and similar directional terms are not to be construed to limit the scope of the invention in any manner. The words attach, connect, couple, and similar terms with their inflectional morphemes do not necessarily denote direct or immediate connections, but can also include connections through mediate elements or devices.

### Overview

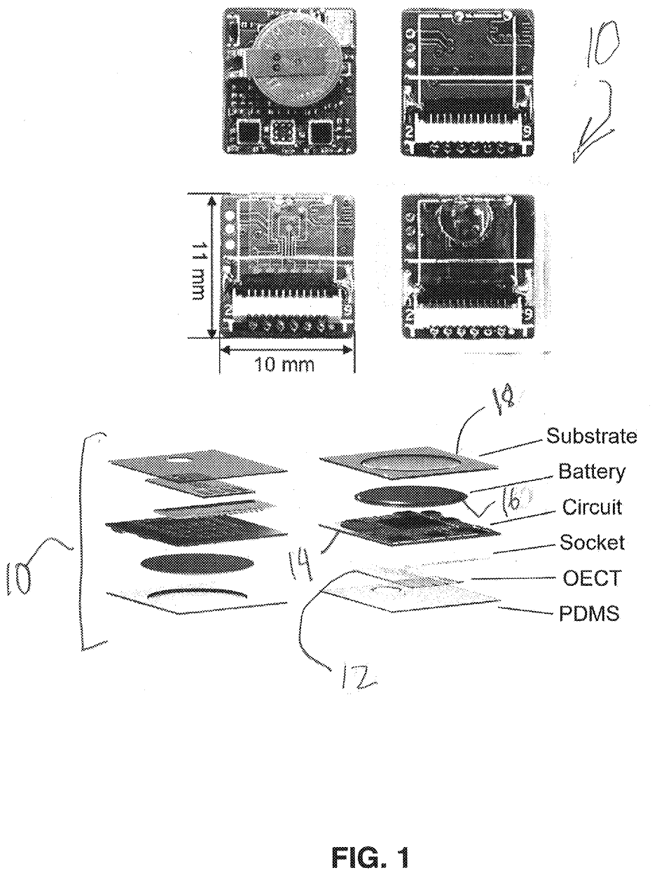

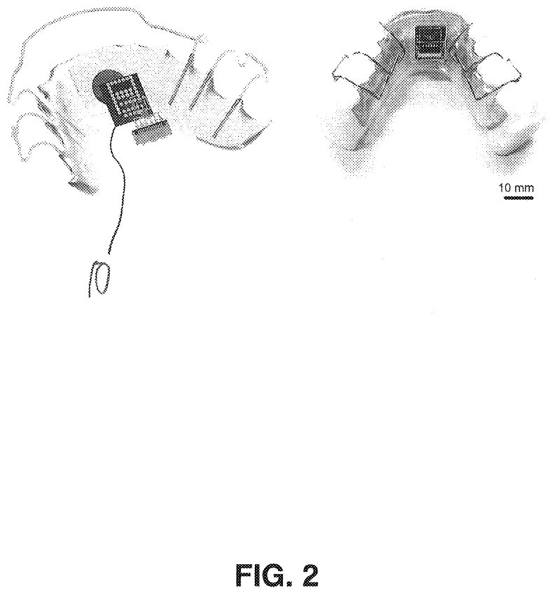

Referring to FIG. 1 and FIG. 2, the invention provides a wearable saliva biosensor system 10 comprising a multi-gate OECT biosensor 20, a wireless integrated circuit 30, a power subsystem 40, and a wearable substrate 50. These components are integrated into a compact, layered platform suitable for placement within the oral cavity. The system is positioned within the mouth in proximity to the sublingual glands, enabling direct, continuous detection of fresh saliva as secreted.

### Multi-Gate OECT Biosensor

The organic semiconductor channel of the OECT biosensor 20 may comprise any suitable p-type or n-type organic semiconductor material, with poly(3,4-ethylenedioxythiophene) polystyrene sulfonate (“PEDOT:PSS”) being a preferred embodiment, other materials such as p-type polymers (e.g., poly(3-hexylthiophene) (P3HT)) or n-type polymers (e.g., pNDI-T2 or BBL) may be utilized to optimize performance for specific analytes.

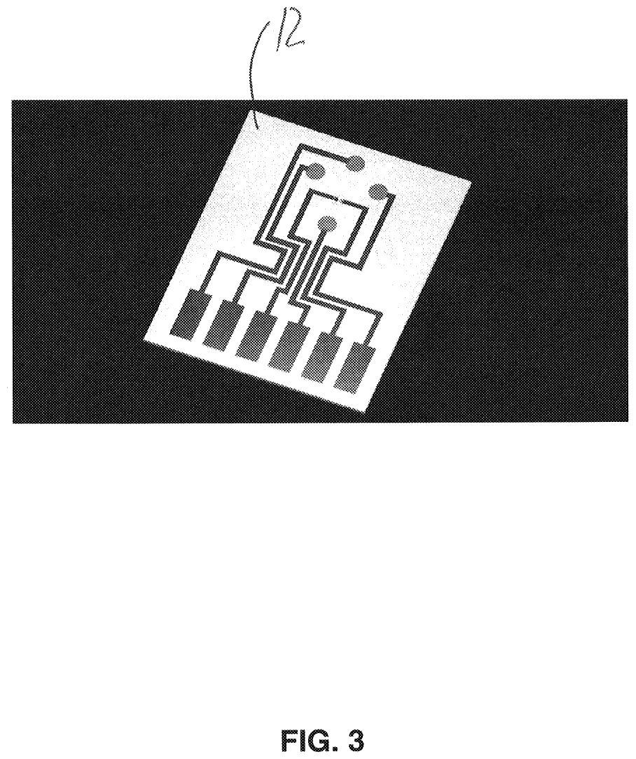

Referring to FIG. 3, the OECT biosensor 20 comprises a patterned PEDOT:PSS film acting as an organic semiconductor channel. Four platinum gate electrodes of identical dimensions are arranged on both sides of the channel. Platinum nanoparticles (“Pt-NPs”) are deposited on the platinum gate electrodes by an electrochemical method. A Nafion layer is then applied over the Pt-NPs by a solution process. Prior to gate electrode functionalization, the OECT biosensor was treated with Plasma, followed by the application of Poly-L-Lysine to the Nafion area. Subsequently, the device was rinsed with deionized water and dried under a nitrogen stream

In one embodiment, one gate electrode is functionalized with a specific enzyme and encapsulated with a layer of chitosan (“CHIT”), while a second gate electrode serves as a control and is coated with CHIT only, without enzyme functionalization. The Nafion and CHIT layers limit the diffusion of large cations and anions—including ascorbic acid, uric acid, lactate, and dopamine—to the gate electrode surface, thereby improving the selectivity of the device through Coulomb interactions.

The differential measurement architecture of the OECT biosensor 20 is central to the selectivity of the device. In operation, the gate voltage is applied simultaneously to both the enzyme-functionalized gate electrode and the control gate electrode. The enzyme-functionalized gate produces a potential change in response to the target metabolite, while the control gate, lacking enzyme functionalization, produces a response attributable solely to non-specific background signal. The difference between the two gate responses is used to derive the biomarker-specific signal, substantially eliminating interference not only from electroactive species present in saliva such as ascorbic acid, uric acid, lactate, and dopamine when those species are not themselves the target analyte, but also from environmental fluctuations including pH shifts, temperature variations, and ionic strength changes inherent in complex biological fluids like saliva.

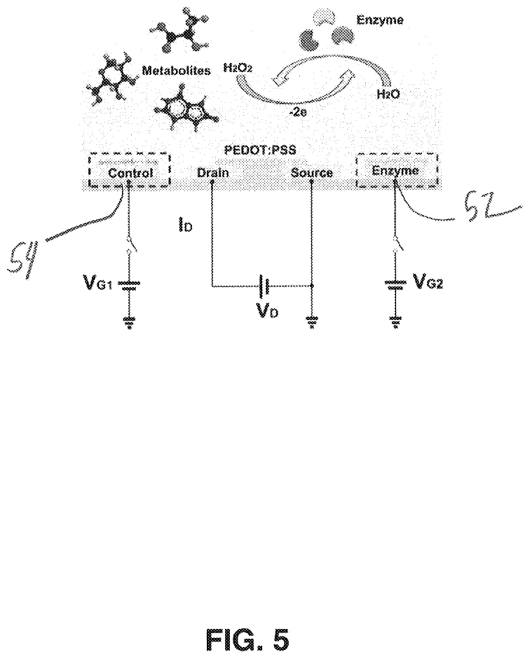

Referring to FIG. 5, when a target metabolite contacts the enzyme-functionalized gate, the enzyme catalyzes a reaction producing hydrogen peroxide (H₂O₂). The H₂O₂ is in turn catalyzed by the Pt-NPs and the Pt gate electrode, causing electron transfer and a change in surface potential at the gate electrode. This potential change modulates the channel current of the OECT, providing a measurable electrical signal proportional to the concentration of the target biomarker. Three separate enzyme-matrix blended solutions—GOx, LOx, and UOx—were formulated. Each enzyme was individually mixed with a stabilizing protein (e.g., BSA) and a cross-linking agent (e.g., glutaraldehyde, or other bioconjugation reagents) to form a stabilized enzymatic matrix.

In one embodiment, glucose oxidase (“GOx”) blended solution is applied to one gate electrode for glucose detection. GOx catalyzes the oxidation of D-glucose, producing gluconolactone and H₂O₂. In another embodiment, lactate oxidase (“LOx”) blended solution is applied for lactate detection, catalyzing the conversion of lactate to pyruvate and H₂O₂. In a further embodiment, uricase (“UOx”) blended solution is applied for uric acid detection, catalyzing the conversion of uric acid to allantoin and H₂O₂. The multi-gate architecture permits simultaneous detection of multiple biomarkers by assigning different enzyme coatings to different gate electrodes within a single device.

The multi-gate architecture of the OECT biosensor 20 enables simultaneous, real-time detection of two or more biomarkers within a single device. In one embodiment, individual gate electrodes are functionalized with glucose oxidase, lactate oxidase, and uricase respectively, enabling concurrent monitoring of glucose, lactate, and uric acid in a single saliva sample without sequential testing or sample manipulation. Each enzyme gate is paired with a dedicated control gate to provide a differential measurement for each target analyte.

### Wireless Integrated Circuit

Referring again to FIG. 1, the wireless integrated circuit 30 comprises an analog front-end (AFE) for signal conditioning, a microcontroller unit (“MCU”), analog-to-digital conversion circuits (“ADC”), digital-to-analog conversion circuits (“DAC”), and a Bluetooth Low Energy (“BLE”) module. In one embodiment, the wireless integrated circuit 30 is configured with a miniaturized form factor suitable for intraoral placement, preferably having a footprint area of less than 1.5 cm² and a thickness of less than 3 mm. In a specific preferred embodiment, the dimensions do not exceed 1 cm by 1 cm by 2 mm.

The system is capable of sampling data at a frequency above 100 Hz, enabling real-time monitoring of biomarker levels. Detection accuracy is within the 1 nA range. Bluetooth transmission and data storage latency is below 10 ms. The channel current response measured by the OECT biosensor 20 is transmitted to a companion mobile application, which converts the measured current into a corresponding biomarker concentration value for display to the user.

The wireless integrated circuit 30 is designed for power efficiency to permit prolonged operation and is robustly constructed to withstand the environmental conditions of the oral cavity.

A companion mobile application installed on a paired smartphone or other external device receives the transmitted data from the wireless integrated circuit 30 via the BLE module. The application processes the received channel current data and applies a calibration algorithm to convert the measured current values into biomarker concentration values expressed in appropriate units. This routine integrates the differential signal processing described in [0022] and may further incorporate temperature or pH compensation to refine accuracy. The processed data is displayed to the user in real time, enabling continuous health monitoring. In one embodiment, the application stores historical biomarker data and presents trend information to the user over time.

### Power Subsystem

The power subsystem 40 may comprise any suitable power source, including without limitation an ultrathin battery, a lithium-ion battery, a button cell, and/or one or more energy harvesting elements.

In one embodiment, the power subsystem 40 further comprises one or more energy harvesting elements, including piezoelectric nanogenerators and/or triboelectric nanogenerators, which convert mechanical energy from oral movement into electrical energy. The incorporation of energy harvesting elements alongside a conventional battery extends the operational life of the system and reduces maintenance requirements.

The OECT biosensor 20, wireless integrated circuit 30, and power subsystem 40 are co-integrated into a unitary sensing platform and encapsulated with a biocompatible encapsulant. In one embodiment the encapsulant is polydimethylsiloxane (“PDMS”), though any biocompatible encapsulant suitable for intraoral use may be employed. The encapsulation provides mechanical protection, waterproofing, and biocompatibility, rendering the integrated platform suitable for continuous intraoral use. The resulting unitary sensing platform is shown in FIG. 1.

### Wearable Substrate

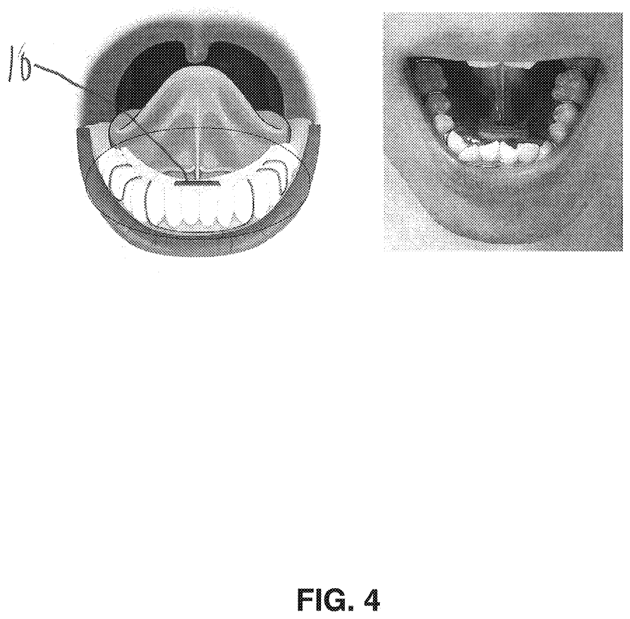

Referring to FIG. 2 and FIG. 4, the wearable substrate 50 is patient-specific and custom-fabricated for each individual user. In one embodiment, the substrate design is based on a three-dimensional scan (e.g., intraoral optical scans or CT data) of the user's dental model, allowing the substrate to be molded to the unique shape and contours of the user's oral cavity. The substrate has a thickness of less than 3 mm, minimizing interference with normal oral function.

The wearable substrate 50 incorporates a self-adaptive fitting mechanism that allows the device to adjust to changes in the user's oral cavity over time, maintaining a secure and comfortable fit. In one embodiment, a design-optimization loop utilizing machine learning algorithms are applied to analyze user feedback and long-term wear data to continuously refine the substrate design for each individual user These algorithms analyze user comfort feedback, pressure distribution data, and long-term wear patterns to iteratively refine the digital CAD model for subsequent substrate iterations, ensuring an optimized fit throughout the treatment or monitoring period.

The substrate material may comprise any biocompatible and flexible material suitable for intraoral use. Suitable substrate materials include, without limitation, elastomers, polydimethylsiloxane (“PDMS”), styrene-ethylene-butylene-styrene (“SEBS”), resins, and rubbers. Dynamic materials including shape-memory polymers and hydrogels may also be employed to enable the substrate to adapt to changes in the user's oral anatomy.

In one embodiment, the wearable substrate 50 is fabricated by the following process. A three-dimensional scan of the user's dental model is obtained using a dental scanning device. The scan data is used to generate a digital model of the substrate geometry customized to the user's oral anatomy. The digital model is used to fabricate the substrate by molding, casting, or additive manufacturing using a biocompatible substrate material. The OECT biosensor 20, wireless integrated circuit 30, and power subsystem 40 are then integrated into the fabricated substrate and sealed with a biocompatible encapsulant. Machine learning algorithms may be applied to refine the digital model over time based on user feedback and long-term wear data, and a revised substrate may be fabricated accordingly.

The wearable substrate 50 is secured to the user's teeth by a robust anchoring element. In one embodiment, the anchoring element is a solid wire or linear structural element attached to one or more teeth. The anchoring element may be fabricated from polymers including without limitation carbon fiber, polyester, and polytetrafluoroethylene (“Teflon”); metal alloys including without limitation stainless steel, cobalt-based alloys, and titanium alloys; and ceramics including biologically inert ceramics such as aluminum oxide, zinc oxide, and silicon carbide, as well as biologically active ceramics such as bioglass, hydroxyapatite, tricalcium phosphate, and calcium phosphate bone cement.

### Placement and Operation

Referring to FIG. 4, the assembled wearable saliva biosensor system 10 is positioned within the oral cavity such that the OECT biosensor 20 is located in proximity to the sublingual glands and the submandibular ducts (Wharton's ducts) that facilitate saliva secretion. This placement enables direct detection of fresh saliva as it is secreted, without the need for external sample collection, and ensures that the measured biomarker concentrations reflect real-time physiological conditions. The proximity of the biosensor to the sublingual ducts ensures that saliva measured by the device is freshly secreted and has not been subject to evaporation, dilution, or contamination by food residue or other oral contents. This distinguishes the system from prior art devices that analyze saliva collected externally, which may not accurately reflect real-time physiological conditions.

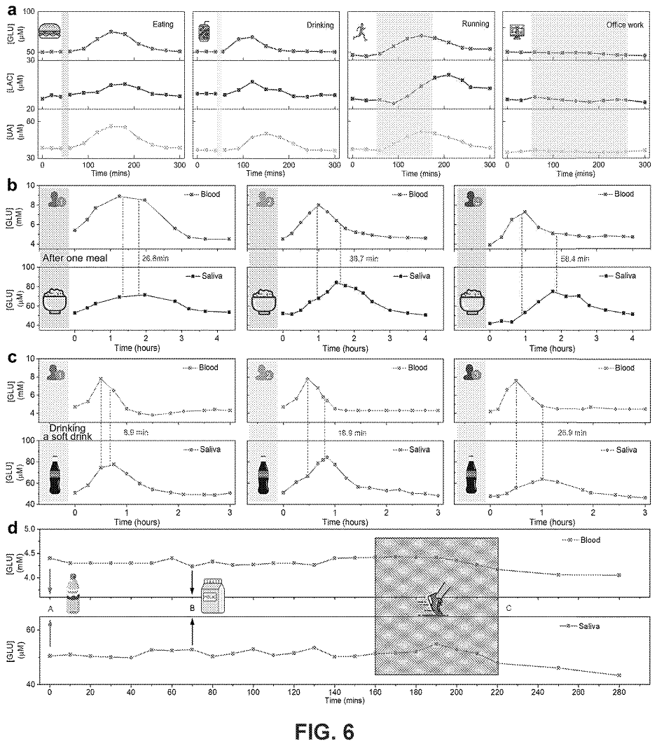

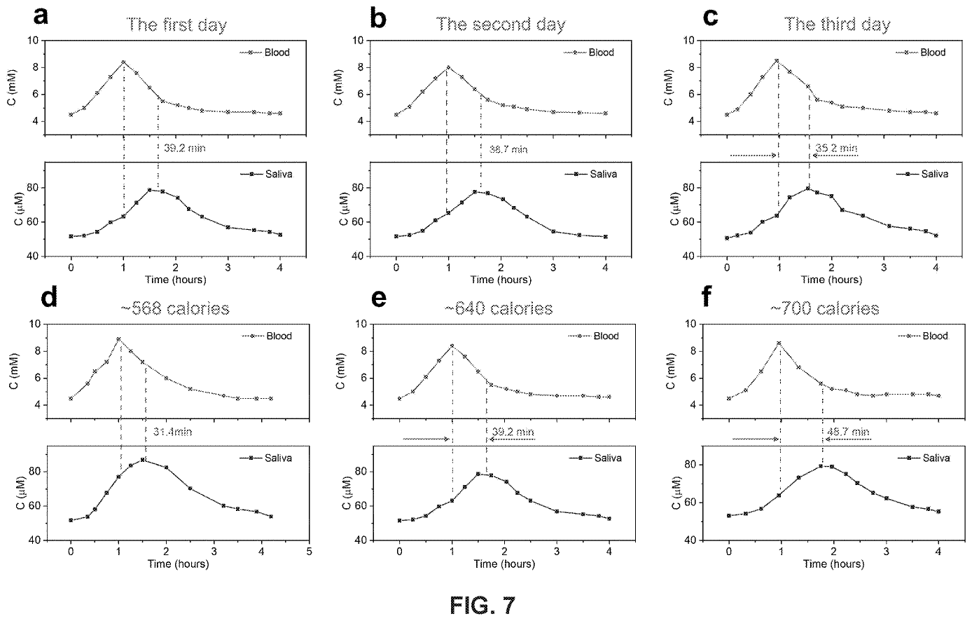

Referring to FIG. 6 and FIG. 7, the wearable saliva biosensor system 10 has been demonstrated to continuously monitor salivary concentrations of glucose, uric acid, and lactate in response to daily activities including eating, drinking, running, and sedentary work. Salivary glucose concentrations increase following food and beverage intake, with a delay of approximately 30 to 50 minutes relative to blood glucose concentration following a meal, and approximately 10 to 30 minutes following consumption of a sugar-containing beverage. Salivary glucose concentrations decline during running or jogging, consistent with observed trends in blood glucose. Consumption of water or milk does not produce significant changes in salivary or blood glucose. These observations confirm that the system provides continuous, real-time, physiologically meaningful monitoring of salivary biomarkers across a range of conditions and activities.

### Data Processing and Output

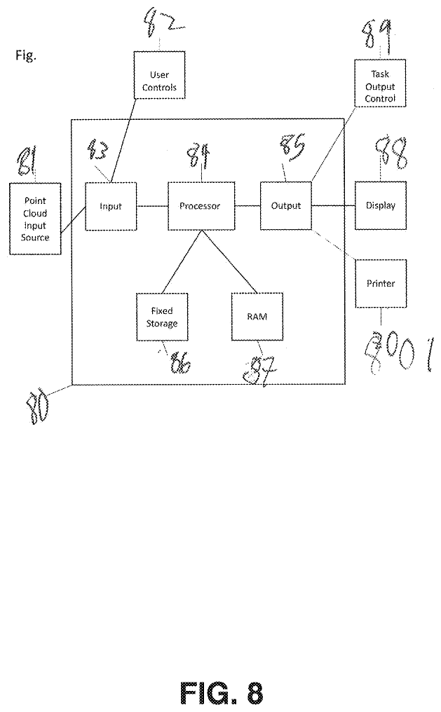

Referring to FIG. 8, the wireless data processing and output components of the invention may be implemented on a general purpose computer 80 or equivalent device. Device 80 includes an input bus 81, user controls 82, processor 83, analog-to-digital conversion circuitry 84, task output control 85, output bus 86, fixed storage 87, RAM 88, display 89, and printer 810. Input bus 81 and output bus 86 can be unified, for example as a USB bus or any other reasonable combination of input and output bus technology. Most of these components are well known in the art and can comprise any desired physical component. In place of fixed storage 87 and RAM 88, a single unified memory device such as Flash RAM can be used. In one embodiment, the functions of device 80 are performed by a smartphone or tablet paired with the wireless integrated circuit 30 via the BLE module, running the companion mobile application described herein.

Although a few embodiments of the present invention have been shown and described, it would be appreciated by those skilled in the art that changes may be made in these embodiments without departing from the principles and spirit of the invention, the scope of which is defined in the claims and their equivalents.

What is claimed is:

1. A wearable saliva biosensor system comprising:
an organic electrochemical transistor (“OECT”) biosensor comprising an organic semiconductor channel, a plurality of gate electrodes, wherein at least one member of the plurality of gate electrodes is functionalized with an enzyme specific to a target biomarker and at least one other member of the plurality of gate electrode serves as a control gate electrode without enzyme functionalization, the biosensor configured to generate a differential electrical signal between the enzyme-functionalized gate electrode and the control gate electrode proportional to a concentration of the target biomarker in a saliva sample;
a wireless integrated circuit configured to sample the differential electrical signal of the OECT biosensor to generate a data stream and transmit the data stream wirelessly to an external device;
a power source integrated with the OECT biosensor and wireless integrated circuit to form a unitary sensing platform; and
a wearable substrate configured for intraoral placement in proximity to a sublingual gland of a user, wherein the unitary sensing platform is integrated into the wearable substrate such that the OECT biosensor is positioned to contact fresh saliva at a point of secretion from the sublingual gland.

2. The system of claim 1, wherein the organic semiconductor channel comprises poly(3,4-ethylenedioxythiophene) polystyrene sulfonate (PEDOT:PSS) and the gates are Pt-NPs.

3. The system of claim 1, wherein at least one of the plurality of gate electrodes are coated with a Nafion layer overlying the platinum nanoparticles and a chitosan layer, and wherein the enzyme-functionalized gate electrode is encapsulated with chitosan.

4. The system of claim 1, wherein the plurality of gate electrodes includes at least four electrodes configured for multi-analyte detection of glucose, lactate, and uric acid.

5. The system of claim 1, wherein two or more of the plurality of gate electrodes are each functionalized with a different enzyme, enabling simultaneous real-time detection of two or more biomarkers in a single saliva sample.

6. The system of claim 1, wherein the wireless integrated circuit comprises a microcontroller, analog-to-digital conversion circuitry, digital-to-analog conversion circuitry, and a Bluetooth module.

7. The system of claim 1, wherein the wireless integrated circuit has a planar form factor not exceeding 1 cm by 1 cm and a height not exceeding 2 mm.

8. The system of claim 1, wherein the power source comprises an ultrathin battery and at least one energy harvesting element selected from a piezoelectric nanogenerator or a triboelectric nanogenerator configured to scavenge energy from oral movement.

9. The system of claim 1, wherein the unitary sensing platform is encapsulated with polydimethylsiloxane.

10. The system of claim 1, wherein the wearable substrate comprises a material selected from the group consisting of elastomers, polydimethylsiloxane, styrene-ethylene-butylene-styrene, resins, rubbers, shape-memory polymers, and hydrogels, and incorporates a self-adaptive fitting mechanism.

11. The system of claim 1, further comprising an anchoring element configured to secure the wearable substrate to one or more teeth of the user, wherein the anchoring element is fabricated from a material selected from the group consisting of polymers, metal alloys, and ceramics.

12. The system of claim 1, further comprising a mobile application configured to receive the wirelessly transmitted data, apply a calibration algorithm to convert measured channel current values into biomarker concentration values, and display the biomarker concentration values to the user in real time.

13. A method for continuous real-time monitoring of salivary biomarkers comprising:
positioning a wearable saliva biosensor system within the oral cavity of a user such that an OECT biosensor of the system is located in proximity to the sublingual glands and contacts fresh saliva at the point of secretion;
simultaneously applying a gate voltage to an enzyme-functionalized gate electrode and a control gate electrode of the OECT biosensor;
measuring a differential electrical signal between the enzyme-functionalized gate electrode and the control gate electrode representing the concentration of a target biomarker in the saliva to create a data stream;
wirelessly transmitting the data stream to an external device; and
converting the data stream into a biomarker concentration value.

14. The method of claim 13, wherein the differential electrical signal is substantially free of interference from non-target electroactive species in the saliva comprising one or more of ascorbic acid, dopamine, and uric acid when uric acid is not the target biomarker.

15. The method of claim 13, wherein two or more biomarkers are monitored simultaneously using two or more enzyme-functionalized gate electrodes bearing different enzyme coatings.

16. The method of claim 13, wherein the biomarker is selected from the group consisting of glucose, lactate, and uric acid, and the differential signal is sampled at a frequency above 100 Hz.

17. The method of claim 13, further comprising harvesting energy from oral movement using at least one energy harvesting element selected from the group consisting of a piezoelectric nanogenerator and a triboelectric nanogenerator to power the biosensor system.

18. The method of claim 13, further comprising storing historical biomarker concentration data and presenting trend information to the user over time.

19. The method of claim 13, wherein the wearable substrate comprises a material selected from the group consisting of elastomers, polydimethylsiloxane, styrene-ethylene-butylene-styrene, resins, rubbers, shape-memory polymers, and hydrogels.

20. The method of claim 13, further comprising securing the wearable substrate to one or more teeth of the user with an anchoring element fabricated from a material selected from the group consisting of polymers, metal alloys, and ceramics.

21. A method of fabricating a customized wearable saliva biosensor system comprising:
obtaining a three-dimensional scan of a dental model of a user's oral anatomy;
generating a digital substrate model from the three-dimensional scan customized to the user's oral anatomy;
fabricating a wearable substrate from the digital substrate model using a biocompatible material;
integrating an OECT biosensor, a wireless integrated circuit, and a power source into the wearable substrate; and
encapsulating the OECT biosensor, wireless integrated circuit, and power source with a biocompatible encapsulant to form a unitary sensing platform within the wearable substrate.

22. The method of claim 21, wherein the OECT biosensor comprises a PEDOT:PSS channel, a plurality of platinum gate electrodes having platinum nanoparticles deposited thereon by an electrochemical method, a Nafion layer overlying the platinum nanoparticles, and a chitosan layer, wherein at least one gate electrode is functionalized with a stabilized enzyme-matrix blend and encapsulated with chitosan and at least one gate electrode serves as a control.

23. The method of claim 21, wherein the enzyme-matrix blend comprises an enzyme selected from the group consisting of glucose oxidase, lactate oxidase, and uricase, mixed with a stabilizing protein and a cross-linking agent.

24. The method of claim 21, wherein the wearable substrate is fabricated by molding, casting, or additive manufacturing.

25. The method of claim 21, wherein the wearable substrate is fabricated to a thickness of less than 3 mm using a biocompatible elastomer or resin.

26. The method of claim 21, further comprising securing the wearable substrate to one or more teeth of the user with an anchoring element.

27. The method of claim 21, further comprising applying machine learning algorithms to user feedback and long-term wear data to generate a revised digital substrate model, and fabricating a revised wearable substrate from the revised digital substrate model.
